SEQUENCE LISTING (4 sequences)

SEQ ID NO: 1 - DNA, 55 bp, Artificial Sequence
  Synthetic nucleic acid sequence for BC2
tagcattccacagacagccctcatagttagcgtaacgatctaaagttttgtcgtc

SEQ ID NO: 2 - RNA, 41 nt, Artificial Sequence
  Synthetic nucleic acid sequence for crRNA1
uaauuucuacuaaguguagaucgucgccguccagcucgacc

SEQ ID NO: 3 - RNA, 41 nt, Artificial Sequence
  Synthetic nucleic acid sequence of crRNA2
uaauuucuacuaaguguagaugaucguuacgcuaacuauga

SEQ ID NO: 4 - RNA, 41 nt, Artificial Sequence
  Synthetic nucleic acid sequence of crRNA3
uaauuucuacuaaguguagauccuggguguuccacagcuga

(19) United States
(12) Patent Application Publication
(10) Pub. No.: US 2022/0381781 A1
(43) Pub. Date: Dec. 1, 2022

(54) METHODS FOR ULTRASENSITIVE DETECTION OF PROTEIN AND CELLULAR BIOMARKERS

(71) Applicant: GEORGIA TECH RESEARCH CORPORATION, ATLANTA, GA (US)

(72) Inventors: Gabriel A. KWONG, Atlanta, GA (US); Shreyas DAHOTRE, Atlanta, GA (US); Yun Min CHANG, Atlanta, GA (US); Adrian HARRIS, Atlanta, GA (US); Fang-Yi SU, Atlanta, GA (US)

(73) Assignee: GEORGIA TECH RESEARCH CORPORATION, ATLANTA, GA (US)

(21) Appl. No.: 17/769,940

(22) PCT Filed: Oct. 16, 2020
(86) PCT No.: PCT/US2020/055904

§ 371 Date: Apr. 18, 2022

Related U.S. Application Data
(60) Provisional application No. 62/915,913, filed on Oct. 16, 2019.
(60) Provisional application No. 63/057,980, filed on Jul. 29, 2020.

(51) Int. Cl. G01N 33/569; G01N 33/543; C12Q 1/6804

(52) U.S. Cl. CPC G01N 33/56983; G01N 33/54388; C12Q 1/6804

(57) ABSTRACT

CRISPR-based diagnostic methods and compositions are provided. One embodiment provides the use of DNA-barcoded antibodies or peptide-MHC (pMHC) tetramers (e.g., Kb-OVA₂₅₇₋₂₆₄, Db-GP100₂₅₋₃₃, Db-GP₃₃₋₄₁) and CRISPR-Cas protein, and a guided DNA endonuclease, to achieve ultrasensitive detection of soluble and cell surface proteins. The disclosed embodiments can use type V: Cas12a; type VI: Cas13a, or Cas13b. Combining DNA encoding with CRISPR-Cas protein recognition is a sensitive system because barcodes can be isothermally amplified and Cas, for example Cas12a, enzymatically cleaves DNA reporters upon barcode detection, providing two rounds of amplification and enabling measurement of protein concentration by sample fluorescence or using by paper-based assays. This platform enables monitoring of protein and cellular biomarkers and further expands the toolbox of CRISPR/Cas-based technologies

### CROSS REFERENCE TO RELATED APPLICATIONS

This application claims priority to and benefit of U.S. Provisional Patent Application No. 62/915,913 filed on Oct. 16, 2019, and to U.S. Provisional Patent Application No. 63/057,980 filed on Jul. 29, 2020, both of which are incorporated by referenced in their entireties.

### STATEMENT REGARDING FEDERALLY SPONSORED RESEARCH

This invention was made with government support under DP2HD0917093 awarded by the National Institutes of Health. The government has certain rights in the invention.

### TECHNICAL FIELD OF THE INVENTION

This invention is generally related to compositions and methods for the ultrasensitive detection and quantification of cells, small molecules, and proteins.

### BACKGROUND OF THE INVENTION

Genetic diagnostics are becoming increasingly more sensitive. For example, CRISPR/Cas systems have revolutionized biomedicine, providing precise approaches for genome editing, transcriptional programming, and epigenetic modulation. Recently, Cas enzymes have been repurposed for CRISPR-based diagnostics, enabling in vitro detection of nucleic acids with high sensitivity. CRISPR-based diagnostics can also be used to detect or diagnose COVID-19 and SARS-CoV-2.

The SARS-CoV-2 pandemic will likely continue through the first half of 2021. With the urgency to provide more test capacity, there is a need for rapid diagnostic capabilities to quickly determine if an individual is infected, and isolate cases more quickly to avoid spread and reduce risks in the workplace. Existing molecular tests for viral infection are predominantly laboratory based, requiring costly reagents and equipment, user training, and on average takes days to return results. This slow sample-to-result time may contribute to the spread of virus as 40-50% of cases may be attributable to presymptomatic or asymptomatic people.

There are currently two major classes of rapid tests. The first class comprises molecular tests that focus on viral RNA detection, which requires RNA isolation from a patient sample followed by isothermal amplification of target viral genes. These approaches can be very sensitive, reaching limit of detection (LoD) down to ˜10 copies per microliter. However, molecular tests are not self-contained devices meaning that the steps of RNA extraction and RNA amplification are performed in a tube or multi-well plate prior to a paper readout. This limits these tests either to testing in centralized laboratories or testing by a trained field-technician. Others (e.g., Abbott) also require a specialized instrument to read out assay.

Self-contained point-of-care (POC) tests based on detection of SARS-CoV-2 antigens have the potential to be faster and less expensive than PCR-based assays that require RNA extraction and specialized equipment. However, current antigen tests lack the ability to amplify detection signals and have poor sensitivity compared to RNA tests. A rapid antigen test that may be performed on site in the workplace or home would support reopening the economy and providing test support to minimize workforce risks.

Therefore it is an object of the invention to provide methods and compositions for nucleic acid diagnostics.

### SUMMARY

CRISPR-based diagnostic methods and compositions are provided. One embodiment provides the use of DNA-barcoded antibodies or peptide-MHC (pMHC) tetramers (e.g., Kb-OVA₂₅₇₋₂₆₄, Db-GP100₂₅₋₃₃, Db-GP₃₃₋₄₁) and CRISPR-Cas protein, and a guided DNA endonuclease, to achieve ultrasensitive detection of soluble and cell surface proteins (FIG. 1). The disclosed embodiments can use type V: Cas12a; type VI: Cas13a, or Cas13b. Combining DNA encoding with CRISPR-Cas protein recognition is a sensitive system because barcodes can be isothermally amplified and Cas, for example Cas12a, enzymatically cleaves DNA reporters upon barcode detection, providing two rounds of amplification and enabling measurement of protein concentration by sample fluorescence or using by paper-based assays. This platform enables monitoring of protein and cellular biomarkers and further expands the toolbox of CRISPR/Cas-based technologies.

Another embodiment provides a self-contained antigen test where direct detection of SARS-CoV-2 antigens is amplified without the need for thermocycling, and test results are read using a paper test strip to allow sample-to-result within an hour.

One embodiment provides a probe for detecting a biological target having a nucleic acid barcode conjugated to a binding moiety through a first end and a detectable signal molecule through a second end, wherein the binding moiety binds a biological target, and wherein at least a portion of the nucleic acid barcode can be can be recognized and bound by a CRISPR-Cas protein. The nucleic acid bar code can be single or double stranded, RNA, DNA, or a combination thereof, or a peptide nucleic acid.

Another embodiment provides a probe for detecting a biological target containing a single-stranded nucleic acid barcode conjugated to a binding moiety through a first end and a detectable signal molecule through a second end, wherein the binding moiety binds a biological target, and wherein at least a portion of the single-stranded nucleic acid barcode can be recognized and bound by a CRISPR-Cas protein. In some embodiments the binding moiety is an antibody or antigen binding fragment thereof, a fusion protein, an aptamer, a peptide-MHC, a lectin, a saccharide, or a multimeric construct. In other embodiments the binding moiety specifically binds to a cell-surface protein, an intracellular component, or a cell surface receptor.

In some embodiments the detectable signal molecule is a fluorescent reporter, a bioluminescent molecule or a mass-tag. The detectable signal molecule can be a quenched fluorescent reporter.

In some embodiments the binding of the CRISPR-Cas protein to the nucleic acid barcode triggers cleavage of a reporter construct which releases of the detectable signal molecule.

In one embodiment, a PolyA spacer is between the binding moiety and the nucleic acid barcode. In some embodiments the nucleic acid barcode is RNA and is recognized and bound by type VI CRISPR-Cas proteins. In another embodiment, the nucleic acid barcode is DNA and is recognized and bound by type V CRISPR-Cas proteins. In some embodiments the biological target is a small molecule, a soluble protein, cancer-specific cell surface marker, or a cell. In some embodiments the nucleic acid barcode is bound by a type V or type VI CRISPR-Cas protein.

Still another embodiment provides a method of ultrasensitive detection and quantification of a target in a biological sample by contacting the sample with an effective amount of at least one probe for detecting a biological target according to claim 1, wherein the binding moiety of the at least one probe for detecting a biological target binds to the target. The method includes the steps of contacting the sample with an amount of a type V or type VI Cas protein effective to cleave the detectable signal molecule from the single-stranded nucleic acid barcode, measuring the detectable signal in the sample; and quantifying the amount of target based on the detectable signal. In some embodiments, the limit of detection is 1 fM of nucleic acid barcode. In some embodiments the method further includes amplification of the nucleic acid barcode prior to contacting the sample with the probe to increase the concentration of nucleic acid barcode. In some embodiments amplification of the nucleic acid barcode comprises loop-mediated isothermal amplification (LAMP), recombinase polymerase amplification (RPA), nucleic acid sequence based amplification (NASBA), strand displacement amplification (SDA), rolling circle amplification (RCA), or helicase dependent amplification (HDA). Measuring the detectable signal can be done by subjecting the sample to mass spectrometry, flow cytometry, or ELISA. In some embodiments, the biological target is a small molecule, a soluble protein, a cell, a specific type of cell, an immune cell, a tumor cell, an antigen-specific cell, or a cancer stem cell. The sample can be a biopsy, tissue, urine, blood, serum, plasma, lymphatic fluid, or biological fluid.

Another embodiment provides a method of multiplexed detection and quantification of a biological target, including the steps of contacting the sample with an effective amount of a first probe for detecting a biological target according to the methods described above, wherein the binding moiety of the first probe for detecting a biological target binds to a first biological target, and wherein the nucleic acid barcode is a ribonucleic acid; contacting the sample with an effective amount of a second probe for detecting a biological target according, wherein the binding moiety of the second probe for detecting a biological target binds to a second biological target, and wherein the nucleic acid barcode is a deoxyribonucleic acid; contacting the sample with an amount of a type V and a type VI Cas protein effective to cleave the detectable signal molecules from the single-stranded nucleic acid barcodes; measuring the detectable signals in the sample; and quantifying the amount of the targets based on the detectable signals. In some embodiments the method includes the amplification of the nucleic acid barcodes prior to contacting the sample with the probes to increase the concentration of nucleic acid barcodes. The amplification of the nucleic acid barcodes can be done by loop-mediated isothermal amplification (LAMP), recombinase polymerase amplification (RPA), nucleic acid sequence based amplification (NASBA), strand displacement amplification (SDA), rolling circle amplification (RCA), or helicase dependent amplification (HDA). In some embodiments, the method includes measuring the detectable signals comprises subjecting the sample to mass spectrometry, flow cytometry, or ELISA. In some embodiments the biological targets are small molecules, soluble proteins, or cells.

Still another embodiment provides a method of multiplexed detection and quantification of a biological target, including the steps of contacting the sample with an effective amount of a first probe for detecting a biological target as described above, wherein the binding moiety of the first probe for detecting a biological target binds to a first biological target, and wherein the nucleic acid barcode comprises half of a sequence that can be recognized and bound by the Cas protein; contacting the sample with an effective amount of a second probe for detecting a biological target as described above, wherein the binding moiety of the second probe for detecting a biological target binds to a second biological target, and wherein the nucleic acid barcode comprises the second half of the sequence that can be recognized and bound by the Cas protein; contacting the sample with an amount of a type V and a type VI Cas protein effective to cleave the detectable signal molecules from the single-stranded nucleic acid barcodes; measuring the detectable signals in the sample; and quantifying the amount of the targets based on the detectable signals, wherein the nucleic acid barcodes are recognized by Cas protein only when the two probes are within close proximity to one another.

Still another embodiment provides a Self-contained Lateral Flow Assay (LFA) or a kit containing a housing having a first and second opening and optional three of four openings, a LFA test strip within the housing, wherein the LFA test strip includes a sample pad exposed to the first opening to receive a sample and reagents, wherein the sample pad includes immobilized pre-adsorbed antibodies that specifically bind an analyte and are conjugated with a DNA barcode; a virus protein capture region exposed to the second opening, wherein the virus protein capture region comprises pre-adsorbed antibodies conjugated on surface of the LFA test strip that specifically bind an analyte; a control region exposed to the second opening comprising preadsorbed antibodies that specifically bind to a binding moiety, and a detection region exposed to the second opening comprising preabsorbed antibodies that specifically bind the detection antibodies; and enzymatic amplification reagents including a nucleic acid guided endonuclease, DNA-reporter conjugates, and detection antibodies. The sample can saliva, blood, mucus, nasal swab with or without viral transport medium, sputum, bronchoalveolar lavage fluid, serum. or combinations thereof. The analyte can be a protein, peptide, antibody, cell, microorganism, virus, an antigen, or combinations thereof. In some embodiments, the analyte is a virus protein, for example a coronavirus protein including but not limited to SARS-COV-2 protein. In some embodiments the proteins is SARS-COV-2 spike protein. In some embodiments the DNA-reporter conjugate comprises two different binding moieties on either end, wherein the DNA of the DNA-reporter is cleavable by the nucleic acid guided endonuclease including but not limited to a Cas endonuclease. The Cas endonuclease can be Cas12a or a variant thereof. In some embodiments the binding moiety is biotin and the detectable agent is a fluorophore. In some embodiments, the detection antibody is an anti-fluorophore antibody conjugated with a gold nanoparticle.

Another embodiment provides a method for detecting an analyte including the steps of combining a sample obtained from a subject with a lysis buffer to form a treated sample and adding an aliquot of the treated sample to the sample pad of the LFA described herein; adding an enzymatic amplification solution comprising a nucleic acid guide endonuclease, cleavable DNA having a different binding moiety on either end, and detection antibodies to the sample pad; and visually detecting the detection antibodies, wherein detection of the detection antibodies in the detection region indicates the presence of the analyte in the sample. In some embodiments, the nucleic acid guide endonuclease is a Cas endonuclease, including but not limited to Cas12a. the analyte is a protein, peptide, antibody, cell, microorganism, virus, an antigen, a viral protein, a SARS-COV-2 protein or a SARS-COV-2 protein is a spike protein. In some embodiments the detection antibody comprises an anti-fluorophore antibody conjugated with a gold nanoparticle. In some embodiments, the visual detection is by the naked eye.

Still another embodiment provides a method for detecting an analyte in a sample including the steps of treating the sample with a lysis buffer; adding DNA-barcoded antibodies that specifically bind the analyte to the treated sample; adding an amplification solution directly to the treated sample, wherein the amplification solution comprises hybridization chain reaction (HCR) hairpin nucleic acids labeled with a detectable label and wherein the DNA-barcode hybridizes to the HCR hairpin nucleic acids; and applying the treated sample to the LFA described above. The method further includes visually detecting a signal in the detection region, wherein detection of the signal in the detection region indicates the presence of the analyte in the sample. The analyte can be a protein, peptide, antibody, cell, microorganism, virus, an antigen, a viral protein, SARS-COV-2 protein, or a SARS-COV-2 protein is a spike protein.

Another embodiment provides a method for detecting an analyte in a sample including the steps of adding a lysis buffer to the sample; adding aliquots of the sample to multiple wells of a multi-well plate, wherein the multi-well plate has antibodies that specifically bind to the analyte bound to the wells of the plate; optionally washing the multi-well plate to remove unbound analyte; adding a second solution to the multi-well plate comprising a guide strand endonuclease, crRNA, and DNA-reporter construct to amplify captured antigens; inserting the LFA described herein into each well of the multi-well plate; wherein a detectable signal in the detection region of the LFA indicates the presence of the analyte in the sample. In some embodiments, the sample is selected from the group consisting of saliva, blood, and serum. The analyte can be a protein, peptide, antibody, cell, microorganism, virus, an antigen, a virus protein, a coronavirus protein, a SARS-COV-2 protein, or a SARS-COV-2 spike protein. In some embodiments the DNA-reporter construct includes a fluorophore conjugated to one end and a binding moiety conjugated to the other end, wherein the DNA of the DNA-reporter is cleavable by the nucleic acid guide endonuclease. In some embodiments the binding moiety is biotin. In some embodiments the nucleic acid guide endonuclease is a Cas endonuclease, including but not limited to Cas12a. In some embodiments the detection antibody includes an anti-fluorophore antibody conjugated with a gold nanoparticle.

Still another embodiment provides a method for detecting an analyte in a sample including the steps of treating the sample with a lysis buffer; adding DNA-barcoded anti-analyte antibodies that are pre-labeled with a long polymeric chain or that contain a polymer chain whose subunits can be recognized by nanoparticle labeled antibodies to the treated sample; applying the treated sample to the LFA described above.

### BRIEF DESCRIPTION OF THE DRAWINGS

FIGS. 1A-1C are graphic depictions of barcoded antibodies bind to target proteins in solution or on the surface of cells. Cas12a-recognition of the barcode triggers cleavage of a quenched reporter, enabling detection by sample fluorescence.

FIG. 2A is a line graph of background subtracted fluorescence versus time (min.) of Cas12a complexed with crRNA specific for BC1, BC2, or BC3 incubated with a DNA reporter containing a fluorophore-quencher and BC2. FIG. 2B is a bar graph of maximum fluorescence for crRNA1, crRNA2, or crRNA3 with BC2. An increase in sample fluorescence was only observed in the crRNA2+BC2 sample, indicating that specific target recognition is required for collateral cleavage activity.

FIG. 3 is a bar graph of cleavage velocity (RFU/min) versus concentration. The sensitivity of Cas12a detection for free target DNA in solution is 100 pM.

FIG. 4 is a bar graph of maximum fluorescence versus time (min.). Samples containing no target DNA were isothermally amplified by RPA for various durations. An optimal amplification time of 15 min at 37° C. was performed to avoid detection of non-specific targets.

FIG. 5 shows results from isothermal amplification by RPA prior to detection by Cas12a improves sensitivity by 4 orders of magnitude to 1 fM. FIG. 5 is a bar graph of cleavage velocity (RFU/min) versus concentration (pM).

FIG. 6A is a line graph of background subtracted fluorescence versus Time (min.). FIG. 6B is a bar graph of maximum fluorescence for No BC or barcodes containing varying polyA spacer lengths 5′ of the Cas12a recognition site (0A, 10A, 20A, and 30A, respectively). FIG. 6C is a bar graph of cleavage velocity (RFU/min.) for No BC, 0A, 10A, 20A, and 30A. DNA barcodes containing polyA spacers of varying lengths upstream of the recognition motif were conjugated to anti-mouse CD4. Equimolar concentrations of Ab-DNA conjugates were assayed by Cas12a and no significant difference in signal was observed for different polyA spacer lengths.

FIGS. 7A-7D show results from DNA barcodes that were conjugated to anti-human IL-2 and were used to perform an ELISA on recombinant IL-2. Signal amplification by Cas12a-mediated cleavage of fluorescent reporters resulted in an identical assay range (bottom) compared to a conventional ELISA using HRP (top). FIG. 7A is a schematic diagram of one embodiment. FIG. 7B is a graph of absorbance at 450 nm versus concentration (pg/mL). FIG. 7C is a schematic diagram of another embodiment. FIG. 7D is a graph of cleavage velocity (RFU/min.) versus concentration (pg/mL).

FIGS. 8A-8B show results from DNA barcodes containing a terminal Cy5 fluorophore that were conjugated to anti-mouse CD8. Mouse CD8 cells and stained with barcoded antibody and assayed with or without Cas12a complexed with either non-target or target crRNA. When cells were incubated with Cas12a with the correct target crRNA, a decrease in Cy5 fluorescence was observed, indicating that Cy5 was cleaved from cells. FIG. 8A is a schematic diagram of one embodiment. FIG. 8B is a histogram of Cy5 intensity for cells stained with barcoded antibody before (−) and after (+) addition of Cas12a complexed with non-target (grey) or target (blue) crRNA.

FIG. 9 shows results from mouse splenocytes that were stained with DNA barcoded anti-CD4 and serially diluted. A Cas12a reporter assay was performed on each sample and cell count was resolved across several orders-of-magnitude with high sensitivity. FIG. 9 is a bar graph of cleavage velocity (RFU/min.) versus cell count.

FIG. 10 shows results from P14 and pmel TCR transgenic cells that were stained with P14 targeting barcoded pMHC tetramers. Cas12a reporter assay correctly differentiates antigen-specificity across four orders-of-magnitude.

FIG. 11 is a table showing Cas12a and Cas13a enzymes can be used in conjunction to recognize orthogonal DNA and RNA barcodes on separate targeting ligands. Recognition of both barcodes is necessary for a cleavage of a chimeric DNA-RNA reporter.

FIG. 12 is a table showing DNA barcodes on separate targeting ligands anneal when in close proximity on the surface of a cell, presenting a motif that is recognized by Cas12a. The complementary length between one half of the split barcode and the crRNA is insufficient to trigger collateral cleavage.

FIG. 13 is a schematic diagram showing a self-contained POC antigen test with signal amplification. Amplyfy™ includes three steps. Capture (left): Viral antigens are captured by immobilized antibodies and then tagged with a DNA barcode by detection antibodies. Amplification (middle): DNA barcodes are isothermally amplified by Cas12a cleavage of DNA reporters. Detection (right): Cleaved and uncleaved DNA reporters are detected downstream by test and control lines, respectively.

FIG. 14 is a schematic diagram showing a point of care (POC) antigen test with signal amplification in sample collection tube. Capture (left): Samples are eluted into a collection tube, lysed, and immediately captured with DNA barcoded antibodies. Amplification (middle): An amplification solution containing HCR hairpins is added to isothermally amplify detection signals. Detection (right): Sample is applied to the LFA and signal is detected at the test line.

FIG. 15 is a schematic diagram showing POC antigen test using pre-amplified antibodies in sample collection tube. Capture (left): Samples are eluted into a collection tube, lysed, and immediately captured with pre-polymerized antibodies (e.g., antibody labeled with a long PEG chain). Detection (right): Sample is applied to the LFA and signal is detected at the test line.

FIG. 16 is a schematic diagram showing a multi-well Amplyfy™ antigen test for high-throughput testing. Samples are applied to multi-well plates where DNA-barcoded sandwich antibodies allow amplification of detection signals without thermocycling. Results are read by companion LFAs without the need for a plate reader.

FIGS. 17A-17G show results from targeting Cas12a to DNA-barcoded antibodies to detect protein antigens. FIG. 17A is a schematic diagram showing that upon binding to target cDNA, Cas12a-crRNA complexes exhibit collateral cleavage and cleaves fluorescent reporters in solution. FIG. 17B is a bar graph of cleavage velocity (RFU/min.) versus concentration (pM) showing Cas12a cleavage upon binding to free barcodes as monitored by increases in sample fluorescence. FIG. 17C is a bar graph of cleavage velocity (RFU/min.) versus concentration (pM) showing Cas12a cleavage assay on mouse IgG coupled with DNA barcode. FIG. 17D is a schematic diagram showing that in a standard ELISA, the detection antibody is bound by streptavidin-HRP (SA-HRP) which catalyzes the conversion of the substrate TMB to a colored product. FIG. 17E is a graph of absorbance at 450 nm versus log[IL2] (pg/mL) showing results from well absorbance in ELISA for various concentrations of human IL-2. FIG. 17F is a schematic diagram showing that antigen is captured using an antibody sandwich pair with a DNA barcoded-detection antibody. The barcode is amplified by RPA and detected by Cas12a. FIG. 17G is a graph of cleavage velocity (RFU/min.) versus log[IL2] (pg/mL) showing cleavage velocity of Cas12a reporter assay for various concentrations of human IL-2. Data shown as mean ±s.d. n=3.

FIGS. 18A-18C show a multi-well Amplyfy™ test prototype for detecting SARS-CoV-2 spike antigen by LFA readout. FIG. 18A is a schematic diagram showing samples containing recombinant spike protein arrayed in multi-well capture plate. Detection antibodies are added followed by Cas12a amplification. FIG. 18B a photograph of one embodiment of a multi-well plate during final step of a test with an lateral flow assay (LFA) readout. FIG. 18C is a photograph of representative LFA strips showing test and control lines as a function of spike protein concentration.

FIGS. 19A-19B show LAMP amplification improves the LOD of multi-well Amplyfy™. FIG. 19A is a schematic diagram showing samples containing recombinant spike protein arrayed in multi-well capture plate. Detection antibodies are added followed by LAMP amplification and Cas12a amplification. FIG. 19B is a photograph of a representative LFA strips showing test and control lines as a function of spike protein concentration.

FIG. 20 is a photograph showing that Amplyfy™ achieves LODs necessary to detect clinical SARS-CoV-2 viral titers. Serial dilutions of free DNA barcodes were assayed by Amplyfy™. Shown are representative LFA strips showing test and control lines. Barcode concentration was converted to viral titer assuming 1 antibody-DNA conjugate per Spike protein and 60 Spike proteins per virus.

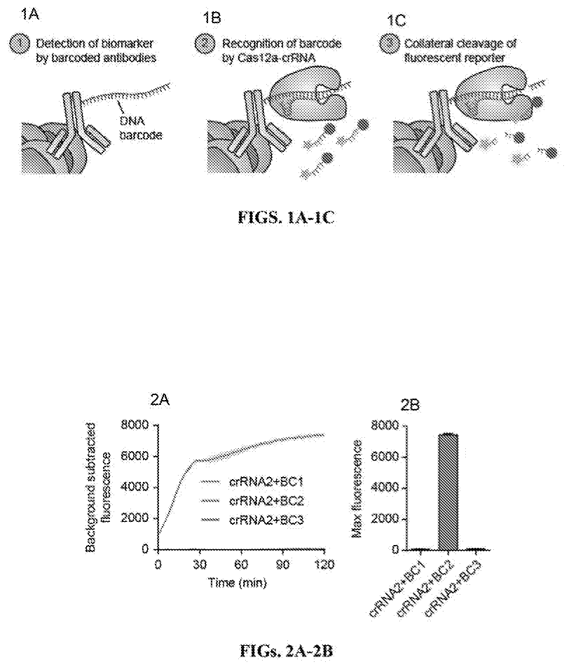

### DETAILED DESCRIPTION OF THE INVENTION

### I. Definitions

It should be appreciated that this disclosure is not limited to the compositions and methods described herein as well as the experimental conditions described, as such may vary. It is also to be understood that the terminology used herein is for the purpose of describing certain embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Methods For Ultrasensitive Detection Of Protein And Cellular Biomarkers By Cas Effector Proteins

A. Collateral Cleavage of Nucleic Acid Reporters by CRISPR/Cas Effector Proteins

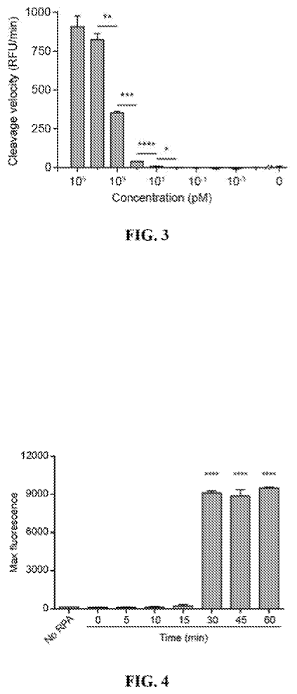

CRISPR effector proteins complex with guide RNA (gRNA) strands, comprising a CRISPR RNA (crRNA) and in some cases a trans-activating CRISPR RNA (tracrRNA). These activated complexes recognize nucleic acids such as DNA or RNA through specific annealing of the gRNA to the target sequence, initiating cleavage of the bound target. Recently, certain types of Cas proteins (e.g., type V: Cas12a; type VI: Cas13a, Cas13b) have been observed to perform “collateral” cleavage of non-target nucleic acids having no complementarity to the gRNA upon target recognition. To test this promiscuous endonuclease activity, Cas12a was complexed with crRNA specific for BC1, BC2, or BC3 (crRNA1, crRNA2, or crRNA3, respectively) and incubated with Barcode 2 and a labeled single-stranded DNA reporter containing a fluorophore-quencher pair. It was observed an increase in sample fluorescence only in samples containing Barcode 2 with Cas12a-crRNA2, indicating that collateral cleavage activity is dependent on specific recognition of target DNA (FIGS. 2A-2B). To determine the sensitivity of barcode detection by Cas12a, a Cas12a reporter assay was performed using a range of barcode concentrations and found that barcodes could be detected at approximately 100 pM (FIG. 3).

The nucleic acid sequence for BC2 is:

(SEQ ID NO: 1)
TAGCATTCCACAGACAGCCCTCATAGTTAGCGTAACGATCTAAAGTTTT
GTCGTC.

The nucleic acid sequence for crRNA1 is:

(SEQ ID NO: 2)
UAAUUUCUACUAAGUGUAGAUCGUCGCCGUCCAGCUCGACC

The nucleic acid sequence of crRNA2 is:

(SEQ ID NO: 3)
UAAUUUCUACUAAGUGUAGAUGAUCGUUACGCUAACUAUGA

The nucleic acid sequence of crRNA3—

(SEQ ID NO: 4)
UAAUUUCUACUAAGUGUAGAUCCUGGGUGUUCCACAGCUGA

B. Isothermal Amplification of Barcodes by Recombinase Polymerase Amplification (RPA)

To increase the sensitivity of the Cas12a reporter assay, an amplification step was performed to increase the concentration of target barcode in a sample prior to detection by Cas12a. In one embodiment, recombinase polymerase amplification (RPA) was used to isothermally amplify barcodes. An optimal amplification time of 15 minutes at 37° C. was identified based on minimal signal observed in samples containing no target DNA; amplification times longer than 15 minutes resulted in high signal presumably due to non-specific amplification of background DNA (FIG. 4). A ladder of barcode concentrations were used, amplified each sample by RPA, and assayed barcode concentration by Cas12a. It was found that the limit-of-detection was increased 10,000-fold, permitting detection of target barcodes at 1 fM (FIG. 5).

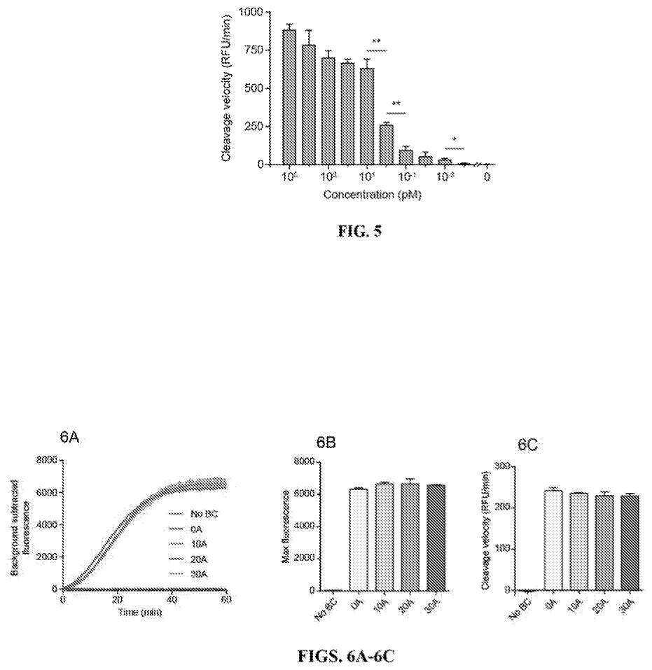

C. Synthesizing nucleic acid-labeled targeting ligands for biomolecule detection Recognition of target biomolecules is initially achieved through binding of an affinity agent coupled to a nucleic acid barcode. The affinity agent can be an antibody or antigen binding fragment thereof, a fusion protein, an aptamer, a peptide-MHC, a lectin, a saccharide, or a multimeric construct. The affinity agent can be conjugated to the nucleic acid barcode using methods known in the art. In one embodiment, a single stranded DNA containing a 5′ or 3′ amine group was conjugated to anti-mouse CD4 using exemplary heterobifunctional linkers 5-HyNic and S-4FB to form a covalent hydrazone bond. Cas12a-crRNA complexes were then validated to retain the ability to recognize target DNA coupled to antibodies and mediate collateral cleavage of a fluorescent reporter, probing a range of polyA spacer lengths between the protein and the target sequence to minimize steric hindrance of Cas12a binding. Collateral cleavage of the reporter was observed only in the presence of antibody-DNA conjugates and found that all barcode lengths triggered reporter cleavage with similar velocity (FIGS. 6A-6C).

In one embodiment, the binding moiety specifically binds a target, for example a cancer specific cell surface marker such as but not limited to PDGF, nucleolin, P-selectin, EpCAM, CD44, Mucin, AXL, PSMA, ICAM-1, VCAM-1, transferrin receptor, ErbB2, VEGFR, HIV-1 Tat protein, HIV Nuceocapsid, integrin, Her3, IL-10, anti-NF-KB, Kanamycin A, catenin, ERK2, C-reactive protein, L-tryptophan, SARS Coronavirus, influenza B, thrombin

Hemagglutinin, tumor necrosis factor-alpha, VEGF, streptavidin, Kit-129, HIV Reverse transcriptase, insulin, PSA, RNase H1, Swine influenza A virus, Human neutrophil elastase, anti-IgE, L-selectin, 4-1BB, Tenascin-C, Protein Kinase C, RBP4 , Enterotoxin B, HER2, Hepatocyte growth factor receptor, Hepatitis C, Fibrogen, HGF, IgG, EGFR, survivin, Osteopontin, P-selectin, neurotrophin receptor, interferon-γ, Human matrix metalloprotease 9, Keratinocyte growth factor, MCP-1, von-Willebrand factor, Plasminogen activator inhibitor-1,OX40, CD4, CD3, CD8, Tenascin-C, androgen receptor (AR), androgen receptor splicing variants (ARV7 (AR3), ARV12, ARV3, ARV1, ARV9, ARV2, ARVS/6, ARV8, ARV9, ARV10, ARV11). In another embodiment, the binding moiety targets a cancer specific antigen such as but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44). Additional tumor antigens include but are not limited to alpha-actinin-4, Bcr-Abl fusion protein, Casp-8, beta-catenin, cdc27, cdk4, cdkn2a, coa-1, dekcan fusion protein, EF2, ETV6-AML1 fusion protein, LDLRfucosyltransferaseAS fusion protein, HLA-A2, HLA-A11, hsp70-2, KIAA0205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2,

p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, GAGE-2, p15(58), CEA, RAGE, NY-ESO (LAGE), SCP-1, Hom/Me1-40, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP and TPS.

D. Cas12a Detection of DNA Barcoded Antibodies and pMHC Tetramers Measures Cell Concentration with High Sensitivity

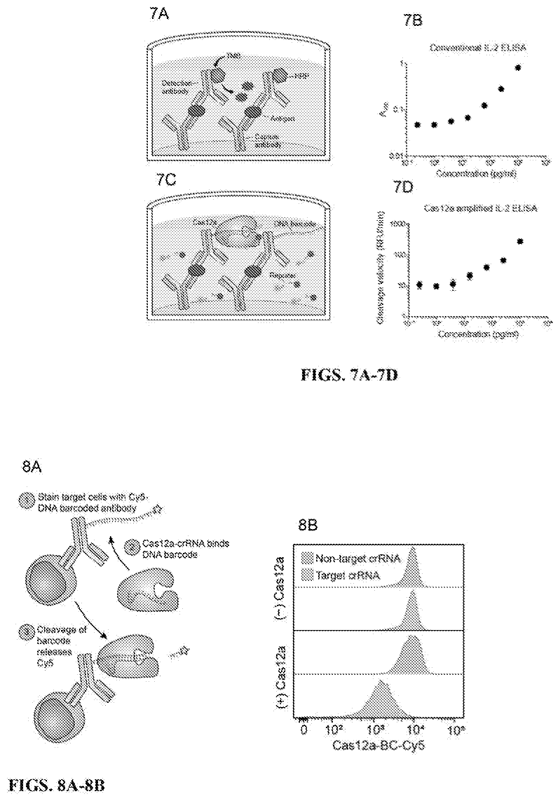

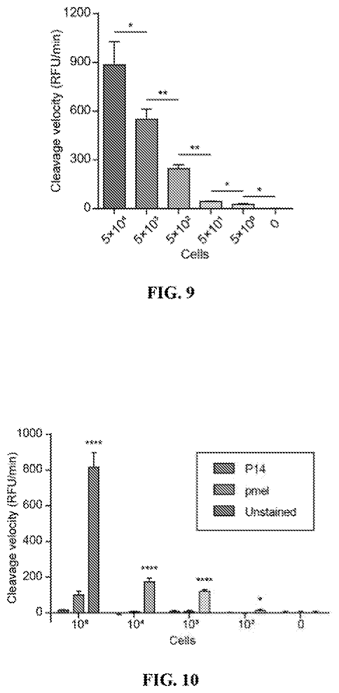

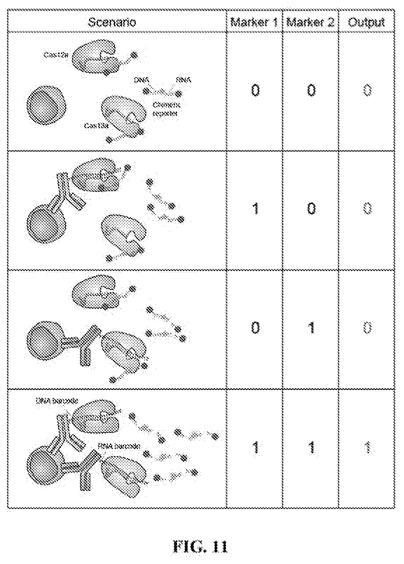

DNA barcoded antibodies were first tested to determine if they could be used to detect soluble proteins. In one implementation, an ELISA was performed for recombinant IL-2 where the detection antibody was labeled with a DNA barcode. Signal amplification was performed by Cas12a-crRNA complexes, resulting in collateral cleavage of a fluorescent reporter. It was found that Cas12a amplified ELISAs had identical sensitivity and assay range compared to conventional ELISAs amplified by HRP (FIG. 7). To test the specificity of Cas12a target cleavage and if cleavage is maintained of the surface of cells, mouse CD8 T cells were stained with Cy5-DNA barcoded anti-CD8 and measured baseline fluorescence by flow cytometry. Cells Cas12a crRNA complexes were then incubated with containing either target or non-target crRNA and found a decrease in cell fluorescence only for cells interrogated with target crRNA (FIGS. 8A-8B). To demonstrate the utility of protein detection using Cas12a, DNA-barcoded anti-CD4 antibodies were used along with amplification by RPA to enumerate CD4 T cells from a mouse spleen, differentiating T cell counts across five orders-of-magnitude with a limit of detection of ˜5 cells (FIG. 9). This approach was then extended to DNA-barcoded pMHC tetramers and found that the antigen-specificity of T cells could be resolved over four orders-of-magnitude (FIG. 10).

E. Logic Gates Using Multiplexed Detection of Nucleic Acid Barcodes

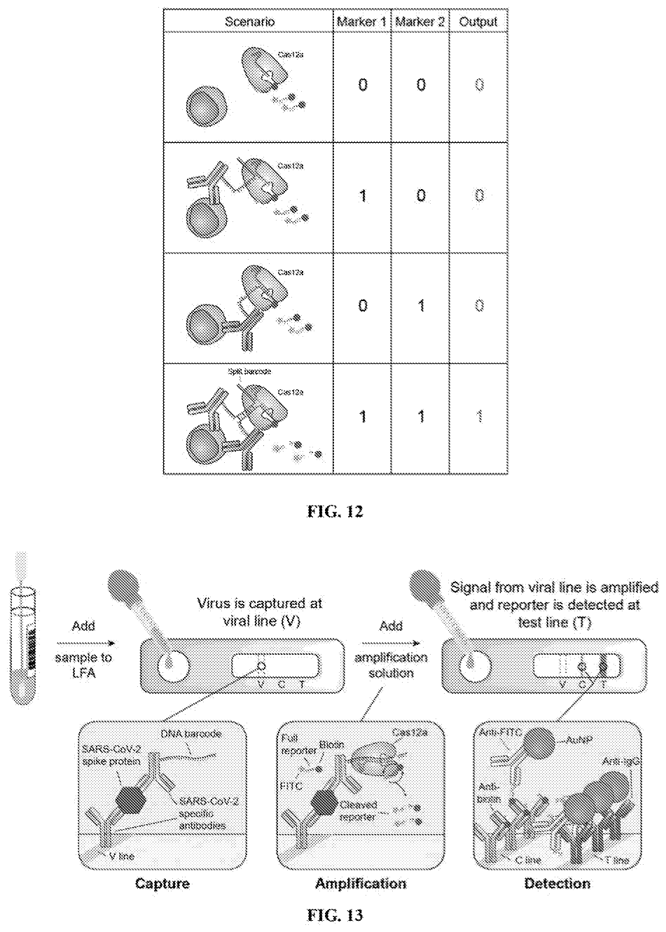

Cell subsets are often defined by the expression of multiple cell surface markers, necessitating the detection of two or more surface receptors. For example, cytotoxic T cells are defined as CD3+CD8+ while helper T cells are CD3+CD4+; in humans, naïve T cells are CD45RA+CCR7+ while terminal effector cells become CD45RA+CCR7—, allowing one to distinguish T cells with distinct functions by marker expression. In the context of disease, subpopulations of cells can be used as clinical biomarkers; for example, CD8+CD27+PD−1—CAR T cells control chronic lymphocytic leukemia (CLL) tumor burden and their presence can be used as a biomarker of treatment response. To achieve logic gates, different barcodes can be used resulting in cleavage of orthogonal reporters. For example, a DNA barcode can be coupled to an affinity agent recognizing the first marker and an RNA barcode can be coupled to an affinity agent recognizing a second marker. Detection of the DNA and RNA barcode by Cas12a and Cas13a, respectively, results in cleavage of a DNA/RNA chimeric reporter and an increase in sample fluorescence (FIG. 11). Other possibilities include the use of nucleic acid circuits that interact on the surface of a cell and only present a domain that is recognized by Cas enzymes upon interactions of barcoded affinity agents sequestered on the cell surface in close proximity (i.e. AND gate). For example, two oligos comprising a “split” barcode are coupled to separate affinity agents; the full barcode is presented when the oligos anneal on the cell surface and can be recognized by Cas enzymes (FIG. 12). Each half of the barcode alone is insufficient to induce collateral cleavage activity of Cas enzymes.

F. Commercial Applications

Platforms based on CRISPR/Cas such as SHERLOCK and DETECTR are an emerging diagnostic tool for the detection of nucleic acids, including viral and bacterial DNA and RNA. These technologies have previously been used to sense bacterial pathogens, detect and differentiate between infections of different flavivirus strains, and detect mutations in cfDNA in non-small cell lung cancer patients. In these applications, the sensing is done through recognition of endogenous DNA or RNA. Here a technology is described that uses CRISPR/Cas to recognize synthetic nucleic acid barcodes coupled to targeting ligands, enabling detection of small molecules, proteins, and cells.

### III. A RAPID TEST FOR SARS-COV-2 WITH ANTIGEN AMPLIFICATION

One embodiment provides a rapid test called Amplyfy™ which is a self-contained LFA device based on isothermal amplification of detection signals to indicate the presence of SARS-CoV-2 antigens in a patient sample (FIG. 13). This approach is analogous to current LFA antigen tests that use sandwich antibodies to capture virions and label them for detection, but instead of a detection antibody labeled with gold nanoparticles (AuNPs) to allow visual detection, this embodiment uses a detection antibody labeled with a DNA barcode. The DNA barcode provides a biochemical handle for amplification of detection signals, such as by Cas12a with a crRNA targeting the barcode. Upon binding, the Cas12a complex exhibits collateral cleavage activity to cleave DNA reporters that can then he visualized downstream by dedicated capture lines. Thus, a single DNA barcode amplifies many DNA reporter fragments. Based on the work to date, it is expected that amplification of antigen detection will increase sensitivity by >100 fold compared to existing antigen tests, while returning results within ˜1 hour after sample collection. Using this mechanism for signal amplification, itis envisioned that there will be multiple embodiments of the test with different intended uses:

A. Self-Contained Lateral Flow Assay

One embodiment provides a fully self-contained LFA where capture and bodies are printed on a LFA test strip, for example a paper test strip consisting of a sample pad, a conjugate pad, a nitrocellulose membrane, and wicking pad (FIG. 13). A patient sample (saliva/swab) is treated with lysis buffer to inactivate CoV-2 virions, followed by direct application of the neutralized sample to the sample pad which contains pre-absorbed detection antibodies conjugated with a DNA barcode. This allows capture and labeling of CoV-2 antigens on a test line by wicking. These steps are identical to that of a traditional LFA. At this stage, a second amplification solution will then be added that contains all the necessary components to amplify captured antigens (i.e., Cas12a, DNA reporter, AuNP-detection antibodies). The detection signals flow downstream and are captured by antibodies printed at test line to allow visualization of test results by eye. In a standard LFA, the AuNPs directly bind to the antigen to provide a visual readout. In this embodiment, the AuNPs bind to amplified reporter to increase assay sensitivity.

B. Tube Amplification with UFA Readout

In this embodiment the amplification step is performed in the patient sample collection tube prior to applying to the LFA (FIG. 14). A patient sample (saliva/swab) is treated with lysis buffer containing a detergent (e.g., 1% Tween-20, 1% Triton X-100, 1% NP-40, 0.5% sodium deoxycholate, or 0.1% SDS) to inactivate CoV-2 virions, followed by direct labeling of the sample with DNA-barcoded antibodies in the same tube. An amplification solution containing all the necessary components (i.e., 1 μM HCR hairpins) is then added to the tube without washing. In HCR, the DNA barcode acts as a trigger by first opening a DNA hairpin (H1), which contains a domain that can subsequently open a second hairpin (H2), which contains a domain that can open a second copy of the first hairpin (H1), and so on. In the resulting hybridization chain reaction, a single DNA initiator results in the synthesis of a DNA polymer composed of many unlocked hairpin strands and thereby containing many copies of fluorophore, providing for strong signal amplification. The amplified sample is then applied to the LFA and signal is visualized at the test line.

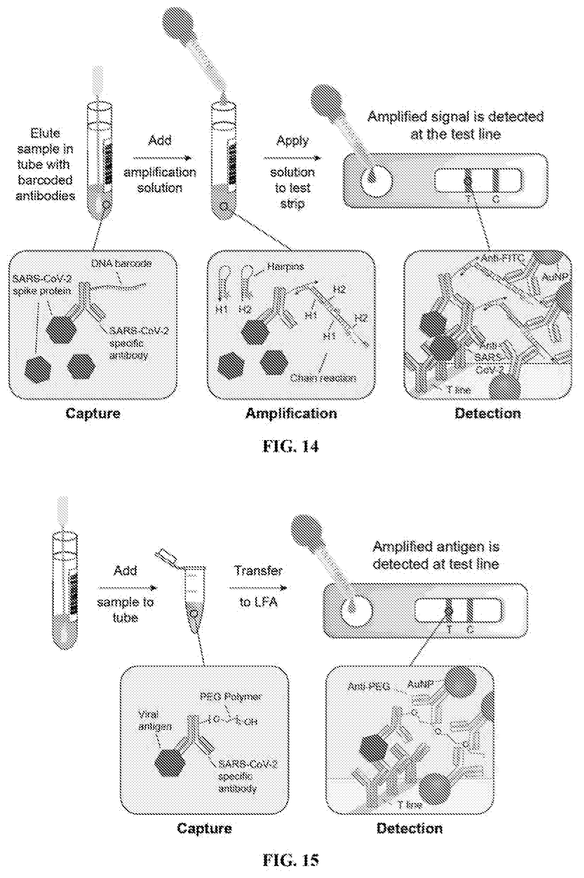

Alternatively, the patient sample is inactivated and then labeled with DNA-barcoded antibodies that have been pre-polymerized (i.e., the HCR reaction in which the DNA barcode triggers the opening of a first hairpin (H1) which contains a domain that opens a second hairpin (H2) which contains a domain that opens a second copy of the first hairpin (H1) and so on occurs during manufacturing of the kit) or with antibodies that contain a polymer chain whose subunits can be recognized by nanoparticle labeled antibodies (FIG. 15). The sample is then applied to the LFA and signal is visualized at the test line. This approach decreases total assay time since amplification occurs prior to the start of the test.

B. Multi-Well Assay with Portable LFA Readout (Non Self-Contained)

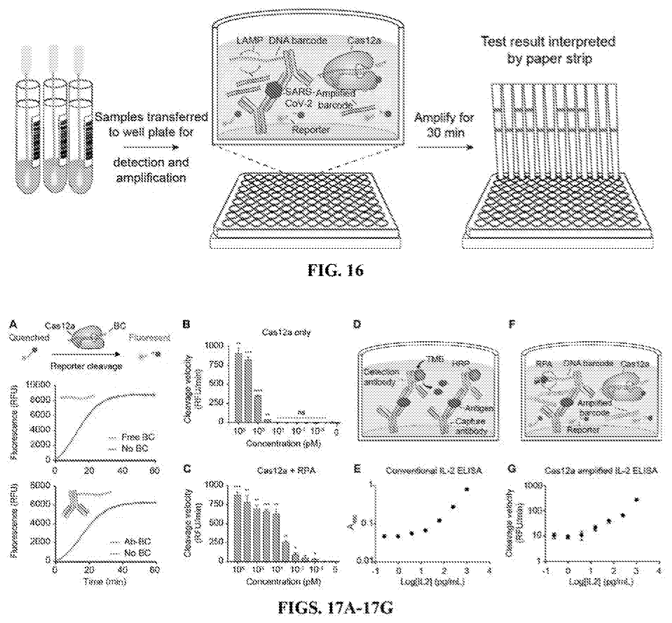

Another embodiment provides is a multi-well plate assay akin to existing molecular tests for SARS-C6V-2 (FIG. 16). The main difference is that RNA extraction and purification of samples will not be required, accelerating test times and reducing the amount of resources required. Here patient samples (saliva/'swab) are first treated with lysis buffer containing a detergent (e.g., 1% Tween-20, 1% Triton X-100, 1% NP-40, 0.5% sodium deoxycholate, or 0.1% SDS) to inactivate CoV-2 virions, followed by direct application of the neutralized sample to multi-well plates to allow capture of CoV-2 antigens by plate-bound antibodies. After washing, a second solution will then be added that contains all the necessary components (e.g., 45 nM Cas12a, 45 nM crRNA, 125 nM DNA reporter to amplify captured antigens. Importantly, this embodiment also allows the inclusion of barcode amplification, such as by Recombinase Polymerase Amplification (RPA) or loop-mediated isothermal amplification (LAMP), prior to Cas12a to further increase sensitivity. The detection signals from each well will be readout out by generic LFA tests printed with universal capture proteins (e.g., streptavidin) that are already commercially available. This embodiment allows high-throughput testing at centralized labs without the need for RNA extraction, a plate reader, and a bespoke LFA. Since the assay is rapid and readout by a LFA, a large proportion of test time is “walk away” time that frees the laboratory technologist for efforts elsewhere.

The chemistry to conjugate DNA barcodes to Abs employs commercial heterobifunctional linkers. This chemistry is robust and previously used to generate DNA-barcoded antibodies and streptavidin. It was validated that DNA oligos coupling to monoclonal antibodies specific for SARS-CoV-2 using gel mobility shift assays, which revealed an average ratio of 1 to 2 DNA oligos per antibody. Purified DNA-barcoded Abs retain their ability to bind to recombinant spike protein compared with unmodified antibody with similar EC50 (˜1 versus 2.5 nM respectively,).

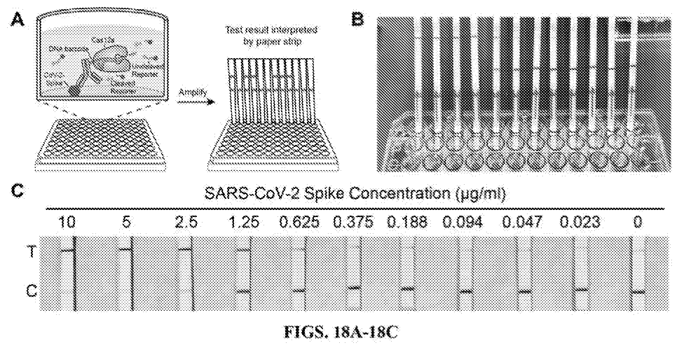

C. Targeting Cas12a to DNA-Barcoded Antibodies to Detect Protein Antigens. (FIG. 17A)

Upon binding to target cDNA, Cas12a-crRNA complexes exhibit collateral cleavage and cleaves fluorescent reporters in solution. (FIG. 17B) Cas12a cleavage upon binding to free barcodes as monitored by increases in sample fluorescence. (FIG. 17C) Cas12a cleavage assay on mouse IgG coupled with DNA barcode. (FIG. 17D) In a standard ELISA, the detection antibody is bound by streptavidin-HRP (SA-HRP) which catalyzes the conversion of the substrate TMB to a colored product. (FIG. 17E) Well absorbance in ELISA for various concentrations of human IL-2. (F) Antigen is captured using an antibody sandwich pair with a DNA barcoded-detection antibody. The barcode is amplified by RPA and detected by Cas12a. (G) Cleavage velocity of Cas12a reporter assay for various concentrations of human IL-2. Data shown as mean ±s.d. n=3.

D. Cas12a Amplification of DNA-barcoded Antibodies to Detect Protein Antigens

The use of crRNA-targeted Cas12a to detect free and antibody-coupled DNA barcodes was validated. Upon Cas12a binding, Cas12a exhibits collateral activity that amplifies the production of cleaved fluorescent reporters, as monitored by fluorimetry (FIG. 17A). It was found that 100 pM DNA barcode was the lowest concentration that could be significantly detected above background (FIG. 17B). To further improve sensitivity, DNA barcode were amplified by recombinase polymerase amplification (RPA) prior to detection by Cas12a. it was found that the lower limit of detection (LoD) improved by 100,000-fold when combining RPA and Cas12a detection compared to Cas12a detection alone to as low as ˜1 fM. This corresponds to an estimated LoD of ˜10 virions/ul which is the lower limit of viral copies observed in the saliva of SARS-CoV-2 infected patients.

Barcoded antibodies were confirmed to retain the ability to bind target antigen in a validated sandwich immunoassay specific for human IL-2. The modified antibody pair was used to capture serial dilutions of recombinant human IL-2 in a 96-well plate, amplified barcodes by RPA, and measured barcode concentration by Cas12a reporter. For benchmarking, the same antibody pair was used to perform a standard ELISA test, which also employs an amplification step through enzymatic conversion of 3,3′,5,5′-Tetramethylbenzidine (TMB) substrate to a colored substance by horse radish peroxidase (HRP) (FIGS. 17D and 17E). It w observed that Cas12a reporter cleavage velocity positively correlated with IL-2 concentration and achieved a similar LoD to ELISA (FIGS. 17F, 17G). These results demonstrate that DNA-barcoded antibodies recognize soluble antigens and allow detection by Cas12a with high sensitivity.

E. Multi-well Amplyfy™ Test Prototype

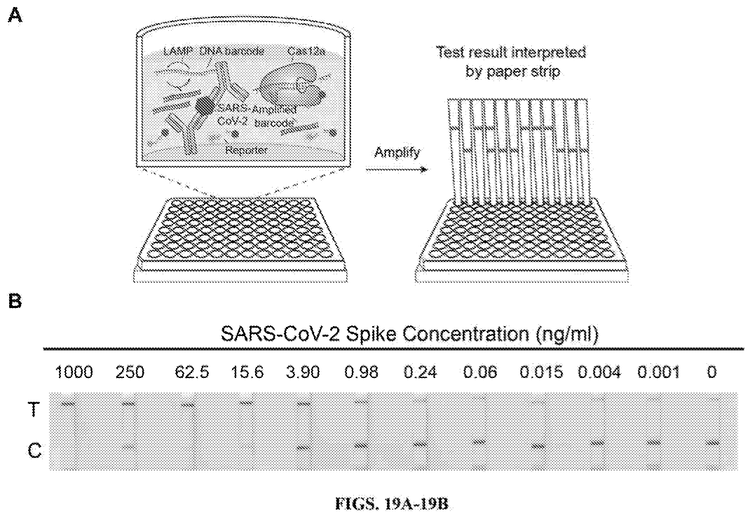

All individual components (i.e., monoclonal antibodies, DNA-barcodes, and Cas12a amplification) were successfully integrated into a multi-well plate prototype of the test assay. This prototype is representative of how one embodiment will be implemented. Here the DNA reporter contains a biotin molecule to allow detection by commercial paper test strips printed with streptavidin without the need for a bespoke LFA printed with SARS-CoV-2-specific antibodies (FIGS. 18A-18C). With this prototype, sample-to-result occurred within 2 hrs with a detection limit of ˜0.625 ug/ml. These parameters are expected only to improve as the test is optimized. For example, this embodiment did not include a step to first amplify the DNA barcode (such as by RPA or LAMP), which is expected to further increase LoD by up to 100,000-fold (FIG. 5). This embodiment is expected to be performed at a CLIA lab or by trained personnel without the need for RNA extraction and a plate reader.

E. LAMP Amplification Improves the LOD of Multi-Well Amplyfy™

A second prototype of the test assay was developed which included isothermal amplification of the DNA barcode by LAMP prior to Cas12a detection to determine the improvement in limit-of-detection. Samples containing recombinant spike protein were arrayed in a multi-well capture plate and sandwiched with a DNA-barcoded antibody (FIG. 19A). DNA barcodes were isothermally amplified by LAMP for 10 minutes followed by addition Cas12a-crRNA complexes and DNA reporters for 1 hour. Test results were read out by LFAs that detect cleaved DNA reporters. With this prototype, the detection limit was ˜1 ng/ml — an improvement of approximately 1000-fold by addition of isothermal amplification by LAMP for 10 minutes (FIG. 19B). Longer LAMP durations are expected to further improve the sensitivity of the test Detection antibodies are added followed by LAMP amplification and Cas12a amplification. FIG. 19B is a photograph of representative LFA strips showing test and control lines as a function of spike protein concentration.

F. Amplyfy™ Achieves LODs necessary to Detect Clinical SARS-CoV-2 Viral Titers

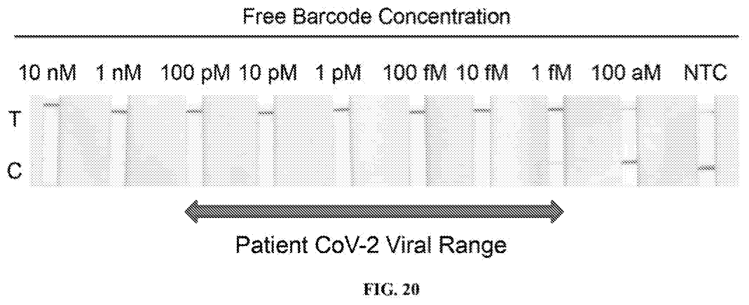

Serial dilutions of free DNA barcodes (as a proxy for live virus) were assayed by methods described herein to demonstrate that these methods can detect clinically relevant viral titers. FIG. 20 shows representative LFA strips showing test and control lines and shows a limit-of-detection of 1 fM. Assuming 1 antibody-DNA conjugate per Spike protein and 60 Spike proteins per virus, this corresponds to a viral titer of ˜10 copies/μl.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A probe for detecting a biological target, comprising:
a nucleic acid barcode conjugated to a binding moiety through a first end and a detectable signal molecule through a second end;
a PolyA spacer between the binding moiety and the nucleic acid barcode;
wherein the binding moiety binds a biological target, and wherein at least a portion of the nucleic acid barcode can be recognized and bound by a CRISPR-Cas protein.

2. The probe of claim 1, wherein the binding moiety is an antibody or antigen binding fragment thereof, a fusion protein, an aptamer, a peptide-MHC, a lectin, a saccharide, or a multimeric construct, wherein the binding moiety specifically binds to a cell-surface protein, an intracellular component, or a cell surface receptor.

3. (canceled)

4. The probe of claim 1, wherein the detectable signal molecule is a quenched fluorescent reporter, a bioluminescent molecule or a mass-tag.

5. (canceled)

6. The probe of claim 1, wherein the binding of the CRISPR-Cas protein to the nucleic acid barcode triggers cleavage of a reporter construct causing release of the detectable signal molecule.

7. (canceled)

8. The probe of claim 1, wherein the nucleic acid barcode is RNA and is recognized and bound by type VI CRISPR-Cas proteins or wherein the nucleic acid barcode is DNA and is recognized and bound by type V CRISPR-Cas proteins.

9-11. (canceled)

12. The probe of claim 1, wherein the biological target is a small molecule, a soluble protein, a cell, or a cancer-specific cell surface marker.

13. A method of ultrasensitive detection and quantification of a target in a biological sample, comprising:
amplifying a nucleic acid barcode to increase the concentration of the nucleic acid barcode;
contacting the sample with an effective amount of at least one probe for detecting a biological target according to claim 1, wherein the binding moiety of the at least one probe for detecting a biological target binds to the target;
contacting the sample with an amount of a type V or type VI Cas protein effective to cleave the detectable signal molecule from the single-stranded nucleic acid barcode;
measuring the detectable signal in the sample wherein the limit of detection is 1 fM of nucleic acid barcode; and
quantifying the amount of target based on the detectable signal.

14-15. (canceled)

16. The method of claim 13, wherein amplification of the nucleic acid barcode comprises loop-mediated isothermal amplification (LAMP), recombinase polymerase amplification (RPA), nucleic acid sequence based amplification (NASBA), strand displacement amplification (SDA), rolling circle amplification (RCA), or helicase dependent amplification (HDA).

17. The method of claim 13, wherein measuring the detectable signal comprises subjecting the sample to mass spectrometry, flow cytometry, a fluorescence plate reader, a hand-held or integrated fluorescence reader or ELISA.

18. The method of claim 13, wherein the biological target is a small molecule, a soluble protein, an immune cell, a tumor cell, an antigen-specific cell, or a cancer stem cell.

19-20. (canceled)

21. The method of claim 13, wherein the sample comprises a biopsy, tissue, urine, blood, serum, plasma, lymphatic fluid, or biological fluid.

22. The method of claim 13, wherein the at least one probe is a first probe for detecting a biological target binding to a first biological target, and wherein the nucleic acid barcode is a ribonucleic acid; or a
second probe for detecting a biological target binds to a second biological target, and wherein the nucleic acid barcode is a deoxyribonucleic acid.

23-26. (canceled)

27. The method of claim 22, wherein the nucleic acid barcode of the first probe comprises the first half of a sequence that can be recognized and bound by the Cas protein,
and wherein the nucleic acid barcode of the second probe comprises the second half of the sequence that can be recognized and bound by the Cas protein.

28. A Self-contained Lateral Flow Assay (LFA) kit comprising:
(1) a housing comprising a first and second opening and optional three of four openings;
a LFA test strip within the housing, wherein the LFA test strip comprises a sample pad exposed to the first opening to receive a sample and reagents, wherein the sample pad comprises immobilized pre-adsorbed antibodies that specifically bind an analyte and are conjugated with a DNA barcode;
a virus protein capture region exposed to the second opening, wherein the virus protein capture region comprises pre-adsorbed antibodies conjugated on surface of the LFA test strip that specifically bind an analyte;
a control region exposed to the second opening comprising preadsorbed antibodies that specifically bind to a binding moiety, and
a detection region exposed to the second opening comprising preabsorbed antibodies that specifically bind at least one detection antibody; and
(2) enzymatic amplification reagents comprising a nucleic acid guided endonuclease, DNA-reporter conjugates comprising two different binding moieties on either end, wherein the DNA of the DNA-reporter conjugate is cleavable by the nucleic acid guided endonuclease, and wherein one binding moiety is biotin and one is a fluorophore.

29. The kit of claim 28, wherein the sample is selected from the group consisting of saliva, blood, mucus, nasal swab with or without viral transport medium, sputum, bronchoalveolar lavage fluid, and serum.

30. The kit of claim 28, wherein the analyte is a protein, peptide, antibody, cell, microorganism, virus, virus protein or an antigen.

31. (canceled)

32. The kit of claim 28, wherein the virus protein is a coronavirus protein.

33. The kit of claim 32, wherein the coronavirus protein is a SARS-COV-2 protein or a SARS-COV-2 spike protein.

34-36. (canceled)

37. The kit of claim 28, wherein the nucleic acid guide endonuclease is a Cas12a endonuclease or a variant thereof.

38. (canceled)

39. The kit of claim 28, wherein the detection antibody comprises an anti-fluorophore antibody conjugated with a gold nanoparticle.

40-70. (canceled)
